(19) United States
(12) Patent Application Publication
(10) Pub. No.: US 2021/0283253 A1
(43) Pub. Date: Sep. 16, 2021

(54) COMPOSITIONS AND METHODS AGAINST P. AERUGINOSA INFECTIONS

(71) Applicant: Aridis Pharmaceuticals, Inc., San Jose, CA (US)

(72) Inventor: Vu Truong-Le, Campbell, CA (US)

(21) Appl. No.: 16/837,858

(22) PCT Filed: Oct. 1, 2018
(86) PCT No.: PCT/US2018/053763

§ 371 Date: Apr. 1, 2020

Related U.S. Application Data
(60) Provisional application No. 62/566,938, filed on Oct. 2, 2017.

(51) Int. Cl. A61K 39/40; C07K 16/12; A61K 31/496; A61K 38/12; A61K 31/546; A61K 31/427

(52) U.S. Cl. CPC A61K 39/40; C07K 16/1214; A61K 31/496; A61K 2039/505; A61K 31/546; A61K 31/427; A61K 38/12

(57) ABSTRACT

A combination of an antibody and other therapeutic that work together in vivo against a pathogenic microbe. The combination can include the antibody with an antibiotic, and/or a therapeutic against a disease. The combination can attack the pathogenic microbe with an efficiency more than either of the components alone, with a synergistic effect, or an effect moderated by one or more modes of action not existing with administration of either the antibody or therapeutic alone.

### FIELD OF THE INVENTION

This invention relates generally to prevention and treatment of microbial infections (such as Pseudomonas aeruginosa infections) and related disorders (e.g., cystic fibrosis) using affinity polypeptides, including human monoclonal antibodies, that bind to the microbe (e.g., at the mucoid exopolysaccharide of P. aeruginosa) in combination with antibiotics.

### BACKGROUND OF THE INVENTION

Pathogenic microbes are often the culprits that ultimately cause mortality in many disease states. Resistance to antibiotics has risen consistently, particularly among Pseudomonas species. Antibodies are an alternative, but have shown an inability to stop chronic bacterial infections, even long after the secondary humoral immune response and cellular response have matured.

For example, P. aeruginosa is an important nosocomial pathogen, which causes different severe acute and chronic infections. Infections with P. aeruginosa are a major health problem for immune-compromised patients and individuals with serious infections such as pneumonia, bacteremia, burn patients, and cystic fibrosis (CF). In fact, given the ubiquitous presence of this microorganism, it appears that most healthy immune systems are quite capable of controlling such infections. However, susceptible individuals, particularly those affected by HIV infection, recipients of transplanted organs, cytotoxic drug addicts, or burn patients with vascular damage hindering localized phagocytosis, frequently suffer from infections caused by this pathogen. Impaired respiratory clearance mechanisms are present in patients with bronchiectasis, a condition that predisposes to colonization and infection by P. aeruginosa. Chronic infections of the respiratory tract are a major cause of the increased morbidity and mortality of individuals with CF. The prevalence of P. aeruginosa infection varies widely among CF centers.

P. aeruginosa, which can survive in different physical conditions, can be responsible for infections determined by medical devices and related to hospital environments. This microorganism is particularly resistant to the current antibiotic arsenal; in fact, it displays intrinsic multidrug resistance (MDR) and has a tremendous capacity to acquire further resistance mechanisms. Recently, widespread extensively drug-resistant P. aeruginosa clones have been reported. Moreover, during chronic infections, the organism can sometimes adopt a mucoid phenotype and/or a biofilm-like mode of growth, resulting in protection from host immune and antibiotic attack.

P. aeruginosa is an opportunistic organism capable of colonizing skin, ear, lung, and bowel. In healthy individuals, such colonization does not normally cause a problem. However, if the individual also has an underlying disorder or condition that compromises their immunity, then infection can be serious. Examples of such disorders or conditions include chemotherapy-induced immunosuppression, diabetes mellitus, cancer, AIDS and cystic fibrosis. It has been estimated that more than 70% of patients with cystic fibrosis are infected with P. aeruginosa. In these patients, P. aeruginosa infection is associated with chronic obstructive bronchitis.

Colonization by P. aeruginosa begins with attachment of the bacterium to epithelial tissues (e.g., lung epithelia). Mucoid strains of P. aeruginosa produce a mucoid exopolysaccharide (i.e., MEP or alginate) which is used by the bacterium throughout the infection. MEP is a polymer of uronic acids.

The bacterium can be relatively resistant to antibiotic therapy due to the double membrane layers and is often evasive to innate and adaptive immune mechanisms, including antibody and complement mediated pathways. MEP is believed to be a contributing factor to immune resistance of the microbe.

DiGiandomenico et al (J. Exp. Med. 2012 Vol. 209 No. 7) identified MAbs that bind three distinct epitopes of the P. aeruginosa exopolysaccharide PsI which is important for P. aeruginosa attachment to mammalian cells and for the formation and maintenance of biofilms produced by non-mucoid and mucoid P. aeruginosa isolates. In 2014 the authors reported the development of a multifunctional bispecific antibody against P. aeruginosa as a potential therapeutic and/or preventive strategy. The bispecific antibody constructs targets both PsI and PcrV.

Secher et al investigated the efficacy of the anti-P. aeruginosa serotype 011 lipopolysaccharide monoclonal antibody Panobacumab in a clinically relevant murine model of neutropenia induced by cyclophosphamide and in combination with meropenem susceptible and meropenem resistant P. aeruginosa induced pneumonia. Panobacumab significantly reduced lung inflammation and enhanced bacterial clearance from the lung of neutropenic host. Further combination of Panobacumab and meropenem had an additive effect and Panobacumab retained activity on a meropenem resistant P. aeruginosa strain (Secher T et al., PLoS ONE 8(9): e73396. doi:10.1371/journal.pone.0073396).

In view of the above, a need exists for more therapeutic options to attack pathogenic microbes, particularly those presenting protective capsules. It would be desirable to have alternative therapeutic compositions, e.g., that present different modes of action, in response to changing defenses of the microbes. The present invention provides these and other features that will be apparent upon review of the following.

### SUMMARY OF THE INVENTION

The compositions of the invention can include affinity polypeptides and therapeutics, e.g., in combination to kill targeted bacteria. The composition can include an isolated polypeptide that selectively binds to a gram negative bacteria, such as P. aeruginosa at an external target (e.g., the mucoid exopolysaccharide), and an antibiotic with some inhibitory effect on the bacteria.

The composition can be, e.g., the qualitative and quantitative combination of at least two compounds selected from isolated polypeptide and antibiotic, whereby the compounds are not necessarily within the same formulation but will be administered adjunctively.

The combination compositions can provide a bactericidal effect in vivo greater than either element alone. In many instances, the particular combination provides an enhanced bactericidal effect or a synergistic effect greater than an additive bactericidal effect. In a particular embodiment, the composition can include an isolated polypeptide that selectively binds to P. aeruginosa mucoid exopolysaccharide, and a bacteriostatic or bactericidal antibiotic effective against gram negative bacteria.

The methods of the invention can include administration of the compositions of the invention, e.g., into a body fluid infected by the bacteria. For example, the compositions can be administered as a powder or solution of the polypeptide and antibiotic by inhalation, by intravenous injection, by intraperitoneal injection, and/or the like. Such methods can provide treatment or prophylaxis against an infection such as, e.g., pneumonia, blood stream infections, keratitis, skin infections, soft tissue infections, meningitis, and/or the like.

The compositions of the inventions can include an antibody (e.g., target binding polypeptide) against a pathogenic microbe, e.g., in combination with another therapeutic agent, such as an antibiotic, another antibody, and/or a therapeutic influencing the local environment or disease state conditions of the infected patient.

In a particular instance, the composition can include an isolated polypeptide that selectively binds to P. aeruginosa mucoid exopolysaccharide, wherein the polypeptide is configured to kill mucoid P. aeruginosa strains without the presence of human complement. For example, the mucoid strain is selected from among strains 2192, M581, 2192, FRDI, PA01, PA101, PA2410, PA27853, PA PGO2338, and 324. Usually, the composition further comprises a bacteriostatic antibiotic effective against gram negative bacteria.

The polypeptide can be an affinity molecule comprising, e.g., heavy and/or light chains of an antibody. In a particular case, the polypeptide has an amino acid sequence selected from the group consisting of SEQ ID NO: 5 heavy chain and/or SEQ ID NO: 7 light chain; or a variant having at least 95% identity to SEQ ID NO: 5 or SEQ ID NO: 7. The polypeptide can be present in the composition at a weight percent ranging from less than about 0.01% to more than 70%, from 0.05% to 50%, from 0.1% to 20%, from 0.1% to 5%, or present at a concentration of at least 0.1% by total solids dry weight. The polypeptide can in many cases kill P. aeruginosa even in the absence of complement.

The second member of the combination can be an antibiotic. For example, the antibiotic can be bacteriostatic or bactericidal against Gram positive or Gram negative target bacteria. Usually, where the bacteria are Gram negative, it can be preferred that the antibiotic is selected from carbapenems, polymyxins, carboxypenicillins, fluoroquinolones, aminoglycosides, and the like. In many cases, the antibiotic is a macrolide, a minocycline, an amikacin, a gentamicin, a kanamycin, a neomycin, a netilmicin, a tobramycin, a paromomycin, a rifaximin, a cephalosporin, an aztreonam, a bacitracin, a sulfonamide, a tetracycline, or the like. Optionally, the antibiotic is bacteriostatic, e.g., selected from the group consisting of a macrolide, minocycline, an amikacin, a gentamicin, a kanamycin, a neomycin, a netilmicin, a tobramycin, a paromomycin, a rifaximin, a cephalosporin, an aztreonam, a bacitracin, a sulfonamide, a tetracycline, and/or the like. For example, preferred antibiotics, from across a wide range of antibiotic families for combination with antibodies, can include ciprofloxacin, colistin, piperacillin, cefepime, tobramycin, meropenem, aztreonam, and/or the like.

The method of preventing growth of pathogenic microbes can include providing a composition of an affinity polypeptide directed to the pathogen and an antibiotic with at least some inhibitory effect against the pathogen. The combination of polypeptide and antibiotic are administered to contact the pathogen, e.g., in a body fluid. For example, a Pseudomonas species can be inhibited or killed by providing an isolated polypeptide that selectively binds to Pseudomonas mucoid exopolysaccharide, providing an antibiotic selected from the group consisting of: a carbapenem, a polymyxin, a carboxypenicillin, a fluoroquinolone, and an aminoglycoside, and contacting the Pseudomonas species in the presence of a body fluid with the polypeptide and antibiotic. The combination of the polypeptide and antibiotic can provide an enhanced effect, an additive effect, or a bactericidal effect greater than either alone.

In many compositions or methods of the inventions, the combination of the polypeptide and antibiotic provides enhancement driven by a mode of action not present with either the antibody or antibiotic alone. Even where the effect is less than an additive effect, the overall effect can be enhanced by one or more interactions between the antibody and antibiotic on P. aeruginosa. The enhanced effect can be the result of interactions reflecting modes of action between the antibody and antibiotic that only exist in the combination. For example, the particular antibiotic could prevent bacterial proliferation sufficiently to enable the antibody to recruit complement deposition and immune cell mediated phagocytosis of P. aeruginosa.

A method of preventing or treating a P. aeruginosa infection can include administering to a subject having or at risk of developing an infection, an isolated polypeptide that selectively binds to P. aeruginosa mucoid exopolysaccharide in combination with a bacteriostatic antibiotic effective against gram negative bacteria.

A method for treating a subject having, or at risk of developing, an infection by P. aeruginosa, including strains such as PA01, PA101, 2192, M581, 2192, FRDI, PA2410, PA27853, PaPGO2338 or 324 s, can include administering to a subject in need of such treatment an isolated polypeptide that selectively binds to P. aeruginosa mucoid exopolysaccharide and configured to kill the P. aeruginosa strains without the presence of complement. Often a bacteriostatic antibiotic effective against gram negative bacteria is co-administered with the polypeptide. Administration can be by, e.g., administering intravenously, subcutaneously, or intramuscularly of a combination of polypeptide and antibiotic in a solution or powder either by inhalation or intradermal administration using a jet injector or a microneedles patch.

The body fluid for the interactions of compound components and the pathogen can be, e.g., blood plasma, respiratory system mucus, synovial fluid, cerebrospinal fluid, gastrointestinal mucus, and/or the like.

The method can treat or prevent an infection, such as, e.g., pneumonia, blood stream infections, keratitis, skin infections, soft tissue infections, and meningitis. The infection can be affecting a patient having an illness, such as cystic fibrosis, COPD, bronchiectasis, pneumonia, and sepsis. The combination of polypeptide and antibiotic can be administered in a powder or solution, e.g., by inhalation, intravenous injection, or intraperitoneal injection. A dose of the combination can range, e.g., from less than about 1 mg peptide per kilogram of patient weight (mg/kg) to 80 mg/kg, from 2 mg/kg to 40 mg/kg, from 3 mg/kg to 20 mg/kg, from 10 mg/kg to 20 mg/kg, or about 10 mg/kg.

The polypeptide can have an amino acid sequence selected from the group consisting of SEQ ID NO: 5 and SEQ ID NO: 7 or a variant having at least 85%, 90% or 95% identity to SEQ ID NO: 5 or SEQ ID NO: 7.

Methods of the invention can include administration to a Pseudomonas infected patient compositions of the anti-MEP antibody Aerucin® (also called F429 or aerubumab) with any of meropenem, colistin, tobramycin, ciproflaxin, piperacillin, cefepime, aztreonam and other antibiotics. The combinations in the compositions can have a greater benefit in treating an infection or inhibiting the Pseudomonas (e.g., in a body fluid) than Aerucin™ or the second component alone (see, e.g., Example 1 and 3, below). The combination can have an additive effect, or a synergistic effect. The antibody and second component can interact with each other, or act in concert on Pseudomonas aeruginosa bacteria, and/or with the patient local environment according to modes of action not existing when either of the components are administered alone.

### Definitions

Before describing the present invention in detail, it is to be understood that this invention is not limited to particular devices or biological systems, which can, of course, vary. It is also to be understood that the terminology used herein is for the purpose of describing particular embodiments only, and is not intended to be limiting. As used in this specification, the singular forms “a”, “an” and “the” include plural referents unless the content clearly dictates otherwise. Thus, for example, reference to “a surface” can include a combination of two or more surfaces; reference to “bacteria” can include mixtures of bacteria, and the like.

Unless defined otherwise, all technical and scientific terms used herein have the same meaning as commonly understood by one of ordinary skill in the art to which the invention pertains. Although any methods and materials similar or equivalent to those described herein can be practiced without undue experimentation based on the present disclosure, preferred materials and methods are described herein. In describing and claiming the present invention, the following terminology will be used in accordance with the definitions set out below.

The term “about” when used before a numerical designation, e.g., temperature, time, amount, concentration, and such other, including a range, indicates approximations which may vary by + or −10%, 5%, or 1%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention.

“Consisting of” shall mean excluding more than trace elements of other ingredients and substantial method steps. Embodiments defined by each of these transition terms are within the scope of this invention.

“Pharmaceutically acceptable composition” refers to a composition that is suitable for administration to a human. Such compositions include various excipients, diluents, carriers, and such other inactive agents well known to the skilled artisan.

“Therapeutically effective amount” or “therapeutic amount” refers to an amount of a drug or an agent that, when administered to a patient suffering from a condition, will have the intended therapeutic effect, e.g., alleviation, amelioration, palliation or elimination of one or more manifestations of the condition in the patient. The therapeutically effective amount will vary depending upon the patient and the condition being treated, the weight and age of the subject, the severity of the condition, the salt, solvate, or derivative of the active drug portion chosen, the particular composition or excipient chosen, the dosing regimen to be followed, timing of administration, the manner of administration and the like, all of which can be determined readily by one of ordinary skill in the art. The full therapeutic effect does not necessarily occur by administration of one dose, and may occur only after administration of a series of doses. Thus, a therapeutically effective amount may be administered in one or more administrations.

“Treatment,” “treating,” and “treat” are defined as acting upon a disease, disorder, or condition with an agent to reduce or ameliorate harmful or any other undesired effects of the disease, disorder, or condition and/or its symptoms. “Treatment,” as used herein, covers the treatment of a patient, and includes: (a) reducing the risk of occurrence of the condition in a patient determined to be predisposed to the condition but not yet diagnosed as having the condition, (b) impeding the development of the condition, and/or (c) relieving the condition, i.e., causing regression of the condition and/or relieving one or more symptoms of the condition. “Treating” or “treatment of” a condition or patient refers to taking steps to obtain beneficial or desired results, including clinical results such as the reduction of symptoms. For purposes of this invention, beneficial or desired clinical results include, but are not limited to: preventing infection of a patient at risk of a microbial (e.g. P. aeruginosa) infection; or reducing the severity of infection by the microbe, e.g., by reducing one or more symptoms, reducing the length of time of infection, etc.

As used herein, the term “patient” refers to a mammal. In a preferred embodiment, the patient is a human.

As used herein, the term “strain” or “Pseudomonas strain” refers to any P. aeruginosa strain and includes, but are not limited to, clinical isolates, variants, mutants, and the like. Strains may be resistant to antibiotics or antibiotic-sensitive. Strains may be of mucoid or non-mucoid phenotypes. In general, strains are distinguishable by mucoid phenotype or one or more genetic mutations, even if such mutation does not confer a different characteristic to the bacterium.

As used herein, the term “antibody construct” refers to an antibody wherein at least a portion of the antibody is derived from an antibody from a (e.g., human) patient who had been exposed to the antigen of interest. An antibody construct may be an entire antibody or a fragment or portion thereof, provided the antibody, fragment, or portion has the recited affinity for, e.g., a targeted microbe antigen. An antibody construct may include heavy and light chain combinations not previously existing in nature. An antibody construct may be fully human, humanized, or chimeric. An antibody construct may comprise amino acid sequences derived from a single patient, multiple patients, and/or known antibody sequences (e.g., a consensus constant region sequence).

As used herein, the term “antibody fragment” refers to any portion of the antibody that recognizes an epitope. Antibody fragments may be glycosylated. By way of non-limiting example, the antibody fragment may be a Fab fragment, a Fab′ fragment, a F(ab′)2 fragment, a Fv fragment, a rIgG fragment, a functional antibody fragment, single chain recombinant forms of the foregoing, and the like. F(ab′)2, Fab, Fab′ and Fv are antigen-binding fragments that can be generated from the variable region of IgG and IgM. They vary in size, valency, and Fc content. The fragments may be generated by any method, including expression of the constituents (e.g., heavy and light chain portions) by a cell or cell line, or multiple cells or cell lines.

As used herein, the term “fully human antibody” refers to an antibody, antibody construct, or antibody fragment consisting entirely of human amino acid sequence. That is, the amino acid sequence of the human monoclonal antibody construct is derived from one or more human cells. Such antibodies may be obtained in different ways. For example, the human monoclonal antibody construct consisting of human amino acid sequence can be obtained from a cell engineered to express the variable region heavy and light chains and/or CDRs from an antibody derived from a human patient (e.g., a patient who had been immunized with P. aeruginosa MEP). Alternatively, the human monoclonal antibody construct can be obtained from one or more hybridomas, e.g., wherein the B-cell is a human B-cell. Alternatively, the human monoclonal antibody construct may be obtained by CDR grafting of the CDR regions, onto available human monoclonal antibodies, thereby producing a human monoclonal antibody. The entirely human amino acid sequence of the human monoclonal antibody construct prevents the occurrence of undesired adverse effects such as rapid clearing, rejection reactions, or anaphylactic shock.

As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis.

As used herein, the term “isolated polypeptides” means that the polypeptides are substantially pure and are essentially free of other substances with which they may be found in nature or in vivo systems to an extent practical and appropriate for their intended use. In particular, the polypeptides are sufficiently pure and are sufficiently free from other biological constituents of their hosts cells so as to be useful in, for example, producing pharmaceutical preparations or sequencing. Because an isolated polypeptide of the invention may be admixed with a pharmaceutically acceptable carrier in a pharmaceutical preparation, the polypeptide may comprise only a small percentage by weight of the preparation. The polypeptide is nonetheless substantially pure in that it has been substantially separated from the substances with which it may be associated in living systems. The isolated polypeptide is e.g. a fully human monoclonal antibody or antibody fragment with a specific affinity for e.g. external targets of gram negative bacteria, such as P. aeruginosa.

As used herein, “synergy” refers to an effect in combination where the end result is greater than the effect obtained with the sum of each of the parts of the combination taken separately. “Complementarity” or “enhancement” refers to, e.g., a combination of two elements that provides an effect greater than either of the elements alone.

Antibiotics, antibodies, and other therapeutics have modes or mechanisms of action recognized in the art. As used herein, a “mode of action” refers to the mechanism of how a therapeutic provides a desired result. A primary mode of action is the mode of action for a therapeutic working on its own, e.g., in a patient. These are generally recognized in the art, e.g., as antibody opsonization or antibiotic inhibition of peptide translation. A secondary mode of action here refers to modes of action, other than primary modes of action, e.g., resulting from interactions between the therapeutics and/or with changes in the patient (e.g., local administration environment) resulting from the presence of the therapeutic combination.

“Bacteremia” or “septicemia” refers to the presence of live bacteria in the bloodstream. Typically in bacteremia or septicemia there is a sufficient number such that bacteria can be cultured from a sample of blood from the patient.

As used herein, “complementarity-determining region (CDR)” refers to the three hypervariable regions in each antibody heavy or light chain that interrupt the four “framework” regions established by the light and heavy chain variable regions. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, for example, a VH CDR3 is located in the variable domain of the heavy chain of the antibody in which it is found, whereas a VL CDR1 is the CDR1 from the variable domain of the light chain of the antibody in which it is found.

The complement system is part of the innate immune system. “Complement” is a part of the immune system that enhances (complements) the ability of antibodies and phagocytic cells to clear microbes and damaged cells from an organism, promotes inflammation, and attacks the pathogen's plasma membrane. The complement system consists of a number of small proteins found in the blood, in general synthesized by the liver, and normally circulating as inactive precursors (pro-proteins). When stimulated by one of several triggers, proteases in the system cleave specific proteins to release cytokines and initiate an amplifying cascade of further cleavages. They account for about 10% of the globulin fraction of blood serum and can serve as opsonins.

The term bacteriostatic, as used herein, refers to antibiotics that stop growth of the target bacteria. Bacteriostatic antibiotics typically inhibit the growth and multiplication of the target bacteria. For example, a bacteriostatic antibiotic may target bacterial ribosomes, thus blocking translation of proteins and stopping growth. Bacteriostatic antibiotics can include, e.g., sulfonamides, macrolides, ethambutol, linezolid, tetracyclines, lincosamides, sulfamethoxazole, chloramphenicol, fusidic acid, trimethoprim, clindamycin, erythromycin, other members of the antibiotic families comprising these antibiotics, and/or the like. In certain embodiments, a bactericidal antibiotic, employed at a minimum inhibitory concentration (MIC) can also function as a bacteriostatic antibiotic in the context of the antibody combinations described herein. In preferred embodiments, the antibody/antibiotic combinations of the invention can comprise bacteriostatic and/or MIC levels of tobramycin, meropenem, ciproflaxin, colistin, piperacillin, cefepim, aztreonam, and/or the like.

### BRIEF DESCRIPTION OF THE DRAWINGS

FIG. 1. Individual counts, group means and standard deviations of CFU in lungs measured 18 hours after inoculation for all the groups. The co-administration of tobramycin and Aerucin reduced the bacterial concentration in the lungs more efficiently compared to singly administered drugs. This indicates an additive effect of tobramycin and Aerucin in this murine model of P. aeruginosa PAO1 pneumonia.

FIG. 2. Individual counts, group means and standard deviations of CFU in lungs measured 18 hours after inoculation for all the groups. The co-administration of Meropenem and Aerucin significantly reduced the bacterial concentration in the lung. This indicates a synergistic effect of Meropenem and Aerucin in this murine model of P. aeruginosa PAO1 pneumonia.

FIG. 3. Evidence of Aerucin mediated killing of P. aeruginosa bacteria in the absence of complement using the OPA assay. The opsonic phagocytosis assay (OPA) measures potency of antibodies based on the ability to initiate and mediate destruction of their specific targets by neutrophils. The assay utilizes purified complement from goat, rabbit, guinea pig or human sera, and neutrophils either isolated from whole blood or from differentiation of parental cell lines. Various concentrations of the antibody of interest, along with controls, are mixed with antigens. Complement serum and neutrophils are then added at certain ratios. After incubation to allow phagocytosis to occur, fractions of remaining antigens are then quantified by plating (bacteria) on agar plates to enumerate bacterial colonies remaining from the titer that was used at the start of the assay reaction.

FIG. 4. Effect of Aerucin in combination with different antibiotics. Aerucin and 5 antibiotics were tested in an in vitro assay (OPA). Average results of three independent experiments are given. Error bars represent standard deviations. The data show additive and/or synergistic effects of the combinations across a diverse array of antibiotics.

### DETAILED DESCRIPTION

The present invention relates generally to an adjunctive therapy of administering, e.g., a monoclonal antibody in combination with a bacteriostatic and/or bactericidal antibiotic for treatment of P. aeruginosa caused infections. In certain embodiments of the compositions administered in the same or separate solutions and methods provided herein, an isolated affinity polypeptide is co-administered with another therapeutic agent. The therapeutic agent may be one used prophylactically or therapeutically, such as an antibiotic or antibody targeting, e.g., P. aeruginosa. When administered in conjunction with an antibiotic, the isolated polypeptide can enhance the cytocidal effect of an antibiotic, e.g., by facilitating entry of the antibiotic into a P. aeruginosa colony. This is especially the case where the peptide is an antibody or an antibody fragment that enhances opsonophagocytosis. Alternatively, the combined therapeutic can be, e.g., an agent used in a treatment for a P. aeruginosa related disorder, such as, e.g., N-acetyl cysteine or DNase, which are used in the treatment of cystic fibrosis. In some embodiments, the treatment methods involve administering synergistic amounts of the isolated polypeptide and the other therapeutic agent.

A number of methods and compositions are discussed in the Summary of the Invention and further details are provided herein and in the Examples section. As would be readily appreciated by the skilled person, the disclosures can be read in combination.

The compositions of the invention are generally combinations of affinity molecules and therapeutics (e.g., administered together or separately). For example, a polypeptide with a specific affinity for a target ligand or antigen on a gram negative bacteria in combination with an antibiotic or opsonin effective against the bacteria. More specific embodiments can include, e.g., a combination of a monoclonal antibody (MAb) against a Pseudomonas and an antibiotic with some activity against the Pseudomonas. In a particular case, the polypeptide can be an antibody against a P. aeruginosa mucoid exopolysaccharide (MEP) in combination with a polymyxin or aminoglycoside antibiotic.

The methods in general are directed to methods of inhibiting or killing bacteria with a combination of the affinity polypeptide with the therapeutic agent. The combination can have a greater inhibition of the bacteria than either of the combined agents individually. Further, the combination can provide an additive or synergistic effect. The combination can attack the bacteria according to the known mode of action for both the antibody and antibiotic and according to different modes of action arising from the presence of both the antibody and antibiotic in the environment of the bacteria. In preferred methods, the combination of agents is administered into a patient body fluid containing the bacteria.

### Compositions for Treating Bacterial Infections

As discussed above, the present compositions include combinations of antibodies against a pathogenic bacterium and a secondary therapeutic, for enhanced combined benefits. The secondary therapeutic can be, e.g., an antibiotic against the pathogen, and/or a therapeutic directed against a disease parameter that facilitates the bacteria pathology.

The combination of the primary antibody and the secondary therapeutic provide benefits over either alone. In fact the benefit can be additive, e.g., where the two elements attack the pathogen based on orthogonal modes of action. Further, the benefit in inhibiting or killing the pathogen can be synergistic, providing benefits greater than the sum of the elements alone, e.g., where one element further sensitizes the pathogen to stronger attack by the other element. For example, where the primary antibody enhances degradation of the pathogen protective capsule, this can have additional benefits of accelerating penetration of the second element to the secondary target in (or on) the pathogen. In another example, synergy can result where the secondary element (e.g., bacteriostatic antibiotic) holds the pathogen in a more sensitive state (e.g., cell cycle or invasive location) or provides kinetics more favorable to the primary antibody.

Another effect of combinations can be a new modal effect. That is, the combination can provide a benefit that is not merely an additive effect of the two elements working in their expected modes. For example, the secondary therapeutic can provide the standard expected benefit of the therapeutic working alone in a first mode of action, while also providing a secondary beneficial effect according to a second different mode of interaction with the first, e.g., antibody element. Further, the combined benefit of the modes may be greater, equal, or less than the mathematical addition of the two elements were they working by standard modes alone; but typically has a benefit greater than either alone.

### I. The Antibody

The primary element in the present combinations is an antibody against the bacterial pathogen. The antibody can be directed to any accessible antigen or epitope in or on the pathogen. The target of the antibody can be a known pathogenic factor, such as a capsule, receptor, flagella, enzyme, or endotoxin. The antibody target can optionally be a pathogen epitope not directly associated with pathology, but having an influence on the effectiveness of the secondary therapeutic element.

In a preferred embodiment, the antibody is directed to an antigen or epitope in or on a Pseudomonas spp. mucoid exopolysaccharide (MEP), lipopolysaccharide (LPS), 0-specific polysaccharide of LPS, H-antigen (flagellar antigens), ferripyochelin receptor protein, and/or the like. A preferred primary antibody target is the MEP, preferably that of P. aeruginosa.

Effective exemplary antibodies against P. aeruginosa preferably contain at least one P. aeruginosa MEP-binding complementarity determining region (CDR). A P. aeruginosa MEP-binding CDR is a CDR derived from one of the antibodies recited herein, namely F428, F429, F431 or COMB. See, e.g., U.S. Pat. No. 7,119,172. Importantly, the presence of a MEP coating around P. aeruginosa bacteria can inhibit antibiotic therapy. The antibodies can be present in a polyclonal sera or produced by molecularly manipulating antibody encoding genes from B cells harvested from human subjects immunized with purified MEP. The recombined immunoglobulin (Ig) genes from these B cells, particularly the variable region genes, can be isolated from the harvested B cells and cloned into an Ig recombination vector that codes for human Ig constant region genes of both heavy and light chains. Using this technique, four novel antibodies that bind to P. aeruginosa MEP and enhance opsonophagocytosis of P. aeruginosa have been identified and synthesized. All the antibody clones are of IgG isotype and they are designated F429, F430, F431, and COMB.

On their own, antibodies against bacterial capsules can have a major influence on the progress of disease, or lack thereof. Capsules typically elicit a relatively weak antibody response and present a surface interfering with cell-based immunity (and we also note interference with antibiotic access to the microbes). Binding of antibodies on capsular polysaccharides can provide both opsonization and cause release of cytokines stimulating passive and active cellular immunity. The ability to provide opsonic antibodies to the site of a P. aeruginosa infection can contribute to the eradication of mucoid P. aeruginosa from, for example, the lungs of chronically colonized cystic fibrosis patients. As used herein, the terms opsonic and opsonophagocytic are used interchangeably to refer to an antibody that is able to induce Fc mediated phagocytosis of an antigen such as a bacterium.

Opsonization assays are standard in the art. Generally such assays measure the amount of bacterial killing in the presence of an antibody, an antigen (expressed on the target bacterial cell), complement, and phagocytic cells. Serum is commonly used as a source of complement, and polymorphonuclear cells are commonly used as a source of phagocytic cells. The target cell source can be prokaryotic (as in the present invention) or eukaryotic, depending upon which cell type expresses the antigen. Cell killing can be measured by viable cell counts prior to and following incubation of the reaction components. Alternatively, cell killing can be quantitated by measuring labeled cell contents in the supernatant of the reaction mixture (i.e., chromium release). Other assays will be apparent to those of skill in the art, having read the present specification, which are useful for determining whether an antibody or antibody fragment that binds to P. aeruginosa MEP also stimulates opsonization and phagocytosis.

The polypeptides e.g., antibodies or antibody fragments, that bind to P. aeruginosa MEP, preferably enhance opsonization and phagocytosis (i.e., opsonophagocytosis) of P. aeruginosa, and as a result are useful to a certain extent in the prevention and therapy of a P. aeruginosa infection in a subject. Opsonization refers to a process by which phagocytosis is facilitated by the deposition of opsonins (e.g., antibody or complement factor C3b) on the antigen. Phagocytosis refers to the process by which phagocytic cells (e.g., macrophages, dendritic cells, and polymorphonuclear leukocytes (PMNL)) engulf material and enclose it within a vacuole (e.g., a phagosome) in their cytoplasm. Thus, antibodies or antibody fragments that enhance opsonization and phagocytosis are antibodies or antibody fragments that recognize and deposit onto an antigen, and in doing so, facilitate the uptake and engulfment of the antigen (and the antigen-bearing substance, e.g., P. aeruginosa bacteria) by phagocytic cells. Generally, in order to enhance phagocytosis and opsonization, the antibody comprises an Fc domain or region. The Fc domain is recognized by Fc receptor bearing cells (e.g., antigen presenting cells such as macrophages, or PMNL). As used herein, “to enhance opsonophagocytosis” means to increase the likelihood that an antigen or an antigen bearing substrate will be recognized and engulfed by a phagocytic cell, via antibody deposition. This enhancement can be measured by reduction in bacterial load in vivo or by bacterial cell killing in vitro using the in vitro methods.

The antibodies are able to bind to mucoid and several non-mucoid P. aeruginosa strains. It is believed that strains characterized as “non-mucoid” still secrete low levels of MEP sufficient for detection by the peptides. The antibodies are capable of mediating opsonic killing of P. aeruginosa isolates from infected mammalian subjects. When used in vivo in murine models of P. aeruginosa infection, the antibodies can provide a degree of protection against P. aeruginosa challenge.

The primary affinity polypeptide elements can comprise regions that bind to MEP. For example, P. aeruginosa MEP-binding regions can be derived from MEP-binding regions of the antibodies, or alternatively, functionally equivalent variants of such regions. Two particular classes of antibody derived P. aeruginosa MEP-binding regions are variable regions and complementarity determining regions (CDRs).

An antibody, as is well known in the art, is an assembly of polypeptide chains linked by disulfide bridges. Two principle polypeptide chains, referred to as the light chain and heavy chain, make up all major structural classes (isotypes) of antibody. Both heavy chains and light chains are further divided into subregions referred to as variable regions and constant regions. In some instances, the polypeptides encompass the antibody heavy and light variable chains of the foregoing antibodies. The heavy chain variable region is a polypeptide which generally ranges from 100 to 150 amino acids in length. The light chain variable region is a polypeptide which generally ranges from 80 to 130 amino acids in length. Identified here are for example four different variable regions, two of which are heavy chain variable regions and two of which are light chain variable regions. SEQ ID NO: 1 and SEQ ID NO: 5 correspond to the nucleotide and amino acid sequence of the heavy chain variable region derived from antibody clones F428 and F429. SEQ ID NO: 2 and SEQ ID NO: 6 correspond to the nucleotide and amino acid sequence of the light chain variable region derived from antibody clones F428 and F431. SEQ ID NO: 3 and SEQ ID NO: 7 correspond to the nucleotide and amino acid sequence of the light chain variable region derived from antibody clone F429 and COMB. SEQ ID NO: 4 and SEQ ID NO: 8 correspond to the nucleotide and amino acid sequence of the heavy chain variable region derived from antibody clone F431 and COMB.

The affinity polypeptides acting as the “primary antibody” of the therapeutic combinations can still be functional with only the complementarity determining regions (i.e., CDRs) of the foregoing variable regions. CDRs of an antibody are the portions of the antibody which are largely responsible for antibody specificity. The CDRs directly interact with the epitope of the antigen. In both the heavy chain and the light chain variable regions of IgG immunoglobulins, there are four framework regions (FR1 through FR4) separated respectively by three complementarity determining regions (CDR1, CDR 2 and CDR3). The framework regions (FRs) maintain the tertiary structure of the paratope, which is the portion of the antibody which is involved in the interaction with the antigen. CDRs, and in particular CDR3, and more particularly heavy chain CDR3, contribute to antibody specificity. Because CDRs, and in particular CDR3, confer antigen specificity on the antibody, these regions may be incorporated into other antibodies or peptides to confer the identical antigen specificity onto that antibody or peptide.

The P. aeruginosa MEP-binding region of the affinity polypeptide can be a P. aeruginosa MEP-binding CDR1, a P. aeruginosa MEP-binding CDR2, and/or a P. aeruginosa MEP-binding CDR3, all of which can be derived from the antibodies and antibody variable chains disclosed herein. A primary affinity polypeptide of the therapeutic combination can have a CDR1 amino acid sequence selected from the group consisting of SEQ ID NO: 21, SEQ ID NO: 24, SEQ ID NO: 27, and SEQ ID NO: 30. A CDR2 amino acid sequence can be selected from the group consisting of SEQ ID NO: 22, SEQ ID NO: 25, SEQ ID NO: 28, and SEQ ID NO: 31. A “P. aeruginosa MEP-binding CDR3” is a CDR3 that binds, preferably specifically, to P. aeruginosa MEP, and is derived from either the heavy or light chain variable regions of the antibodies described herein. It preferably has an amino acid sequence selected from the group consisting of SEQ ID NO: 23, SEQ ID NO: 26, SEQ ID NO: 29, and SEQ ID NO: 32. Further, the CDRs concerned with P. aeruginosa MEPs can include functionally equivalent variants understood by those of skill in the art, such as sequences including conservative substitution variants, as described in greater detail below. In addition, it should be understood that the invention also embraces the exchange of CDRs between the variable regions provided herein. Preferably, a heavy chain CDR can be exchanged with another heavy chain variable region CDR, and likewise, a light chain CDR is exchanged with another light chain variable region CDR.

The antibodies discussed above were discovered from screening B-cells of a highly immunized human subject and selected based on its ability to bind complement leading to complement dependent P. aeruginosa killing. Surprisingly, it was discovered that for a number of P. aeruginosa strains, killing was observed in the absence of complement. For example human P. aeruginosa clinical isolates Pa 2410, Pa 27853 and Pa PGO2338.

### II. Antibiotics of the Compositions

The secondary therapeutic of the combined compositions can be an antibiotic. We have found that certain antibiotics complement the antibody in inhibition and killing of the pathogenic bacteria in surprising ways. Without being committed to a particular theory, we believe binding of the antibody and stimulation of complement deposition can lead to the formation of complement attack complex, which facilitates entry of particular antibiotics such as meropenem but not tobramycin. This could explain why synergism was observed with e.g. the Aerucin/meropenem but not e.g. Aerucin/tobramycin combination. These effects are something more than the standard modes of action known for the antibody or antibiotic acting alone. Further, the combination of Aerucin plus Ciproflaxin also appears to show a clear substantial synergistic effect.

Since antibody immune modulating modes of action lead to bacteria destruction, we have hypothesized that the antibodies would complement well with bacteriostatic antibiotics. An aspect of the invention is, e.g., combination of antibodies against P. aeruginosa (e.g., MEP) with bacteriostatic antibiotics, such as, doxycycline, minocycline, and macrolides (azithromycin, clarithromycin, erythromycin), a, minocycline, an amikacin, a gentamicin, a kanamycin, a neomycin, a netilmicin, a tobramycin, a meropenem, a paromomycin, a rifaximin, a cephalosporin, a piperacillin, a cefepim, an aztreonam, a bacitracin, a sulfonamide, a tetracycline, and the like.

Antibiotics are often described as bactericidal or bacteriostatic, though there is not necessarily a fine line between these effects. Bacteriostatic antibiotics are said to essentially stop the bacteria from metabolizing or multiplying (e.g., blocking transcription of peptides). Of course this can ultimately lead to the death of the bacteria. Bacteriostatic antibiotics typically interfere with DNA replication of protein translation. Bactericidal antibiotics typically interfere with construction of a cell feature, such as the cell wall, destroying viability of the cell.

Antibiotics useful in combination with antibodies against bacteria can include, e.g., bactericidal or bacteriostatic antibiotics. For example, the antibiotics can be aminoglycosides (e.g., gentamicin, amikacin, tobramycin), quinolones (e.g., ciprofloxacin, levofloxacin), cephalosporins (e.g., ceftazidime, cefepime, cefoperazone, cefpirome, ceftobiprole), antipseudomonal penicillins, carboxypenicillins (e.g., carbenicillin and ticarcillin), ureidopenicillins (e.g., mezlocillin, azlocillin, and piperacillin), carbapenems (e.g., meropenem, imipenem, doripenem), polymyxins (e.g., polymyxin B and colistin), and monobactams (e.g., aztreonam).

In the specific case of certain bacteria there may be various levels of resistance to antibiotics. However, complete or partial resistance (e.g., as measured in a Kirby-Bauer disc sensitivity assay—Antibiotic Susceptibility Testing by a Standardized Disk Method; Amer. J. Clin. Path. 45:493-496, 1966) may not correspond to the utility of the antibiotic in combination with an antibody against a particular bacterial pathogen. For example, the zone of inhibition about an antibiotic disc may not correlate to the efficacy of a particular antibody/antibiotic combination. Partial inhibition may consume metabolic resources needed to resist the antibody or select for bacteria less equipped to resist the antibody attack. For example, Pseudomonas are often resistant elimination by certain antibiotics (such as, e.g., kanamycin, moxifloxacin, cefuroxime, cefotaxime, ceftriaxone, ertapenem, and many penicillins), but these antibiotics may still surprisingly complement antibodies against the bacteria.

### III. Methods of Using Therapeutic Combinations

The compositions described above can be used to inhibit or kill pathogens. The combinations of therapeutics with antibodies may attack the pathogen according to the modes of action for each element of the combination. Further, the compositions include new functions (modes of action), e.g., associated with effects each element has on the efficacy of other elements in the composition.

The compositions are typically used in the complex in vivo environment of a mammalian patient. The methods can include prevention or treatment of a disease state caused or exacerbated by a microbial pathogen. The methods can generally include providing a combination composition of the invention (e.g., including an antibody against the pathogen and a complementary therapeutic), and administering the composition to a body fluid or surface so that the combination of the composition can make a multi-pronged attack on the pathogen. Typically, the prongs of attack include more modes of action than would have been provided from the composition elements separately.

The compositions can be used to fight infections caused by any of a variety of microbes, e.g., including fungi and bacteria. For example, the bacteria can be Gram positive or Gram negative bacteria, such as, e.g., Bacillus, Bartonella, Bordetella, Borrelia, Brucella, Campylobacter, Chlamydia, Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Salmonella, Shigella, Staphylococcus, Streptococcus, Treponema, Ureaplasma, Vibrio, and/or Yersinia species.

The methods can be applied to patients having any of various disease states caused by microbes. For example, infectious diseases such as pneumonia (HAP/CAP/VAP), infections associated with cystic fibrosis, COPD, bronchiectasis, blood stream infections, kerititis, skin & soft tissue infections, and/or the like.

The compositions can be administered by a route appropriate to the particular infection. For example, oral administration, gastrointestinal/enteral, central nervous system by injection, topical, enteral, parenteral, oral, peritoneal, sublingual, by inhalation, by injection, and/or the like.

Typically, the composition is injected or dissolved into a body fluid. The antibody can be the first therapeutic element of the composition and will typically bind to an accessible antigen on the pathogen. The antibody can, e.g., opsonize the pathogen, aiding active and passive immune system cells to target and phagocytose the pathogen. The presence of the antibody bound to the pathogen can also provide a substrate initiating the complement cascade, e.g., leading to breaching of the microbe cell wall, and recruitment of additional immune cells to the site. Where the secondary therapeutic is an antibiotic, the metabolism of the pathogen is disrupted in a way that slows or stops growth (bacteriostatic) and/or damages the cell in a way that kills the pathogen.

We note that the primary actions of the adjunctive treatment, either administered as a combined composition in a single solution or sequentially as separate treatments, can also result in secondary modes of action (new routes of attack) that do not exist with the elements alone. For example, the primary actions of a first element can cause changes in the local environment, pathogen surfaces, and/or pathogen metabolism that offer avenues for new and different modes of action for the secondary composition element. The pathogen capsule structure, pathogen permeability, pathogen defenses, local immune system cells, local receptors and cytokines, and local tissue structure can change. These changes can modify the intended mode of action for the secondary composition element and also create new unanticipated interactions that influence the bacterial pathology.

In a particular embodiment, the local environment is the lung of a cystic fibrosis patient and the pathogen is P. areuginosa. The primary therapeutic element of the composition can be an antibody against the P. areuginosa capsule of cell membrane. This antibody on its own can be expected to opsonize the bacteria. If the secondary therapeutic is an antibiotic, it can be expected to stop growth or kill the bacteria. However, the presence of both elements can open the interactions to new modes of action. The binding of the antibody to the capsule or membrane can change the accessibility and permeability of the bacteria to the antibiotic, unexpectedly enhancing effectiveness. The presence of the antibiotic can, e.g., reduce the number of targets, so that limited antibody can work on fewer targets. The antibiotic can disrupt the bacterial structure so that the antibody can present more diverse antigens to incoming B-cells, T-cells, and macrophages. The combination of therapeutic elements creates a network of interactions different from the individual elements alone.

The network of interactions does not necessarily provide a greater effect than the sum of the typical outcomes for the individual therapeutic elements alone. Additive effects may be expected in the art. However, results are surprising in that combinations of therapeutics addressing the same parameter (e.g., bacterial inhibition) have been found not to be additive. Further, it is not considered in the art that the therapeutics have surprise effects not only in the addition of their expected modes of action, but interact to present secondary modes of action that have positive or negative effects on the desired parameter (e.g., killing pathogenic microbes).

### EXAMPLES

The following examples are offered to illustrate, but not to limit the claimed invention.

### Example 1—Anti-Bacterial MAb/Antibiotic Combination in Pneumonia

An anti-Pseudomonas aeruginosa MEP binding antibody (called Aerucin®, or F429, or aerubumab) can be efficacious against acute pneumonia in neutropenic mice, and can have cumulative (or complementary) effects with antibiotics. Aerucin was discovered from screening B-cells of a highly immunized human subject and selected based on its ability to binding complement leading to complement dependent P. aeruginosa killing. But we discovered that for a number of Pa strains, killing was observed in the absence of complement.

Formulations:

1. Aerucin stock formulation: 29 mg/ml in 10 mM Histidine, 150 mM NaCl, 0.02% PS20, pH6
2. Control IgG stock: Human IgG1 lambda from myeloma plasma (Sigma 15029). 1 mg/mL in tris-buffered saline w/o preservatives.
3. Tobramycin stock: Tobramycin sulfate (Sigma T1783)
4. Meropenem stock: Meropenem (Sigma M2574) in Phosphate-buffered Saline prepared with Water for Injection.
5. Vehicle: Phosphate-buffered Saline prepared with Water for Injection

Animals: 8 weeks old female BALB/c Mice (Jackson Laboratories, Sacramento), 5 mice per group. Mice are injected intraperitonealy with 30, 100 or 300 mg/kg in 200 μL of PBS, 2 hours after infection. Control animals receive PBS.

Bacteria: P. aeruginosa strains were obtained from ATCC.

Procedure: An experimental murine model of Pseudomonas aeruginosa

PAO1 pneumonia as used. 5 mice of similar weight per group were tested. The inoculum dose used in the experiments was 9.6×10⁶ cfu/mouse for the Tobramycin study and 1.0×10⁷ cfu/mouse for the Meropenem study, respectively.

Mice are anesthetized i.v. with 200 μL of low dose of ketamine/xylazine (1.25 mg/mL; 0.5 mg/mL). 40 μL of the bacterial solution or the corresponding vehicle solution (isotonic saline) is applied intranasally using an ultra-fine pipette tip.

Antibody Aerucin or vehicle were administered intranasally (IN) in two twelve (12) microliters (μl) volumes (for a total of 24 μl) to ketamine/xylazine anesthetized mice.

Tobramycin or vehicle was administered intraperitoneally (IP) in a 200 microliters (μl) volume to unanesthetized mice.

Meropenem and vehicle were administered subcutaneously (SC) in a 200 microliters (μl) volume to unanesthetized mice.

Mice were sacrificed and the lungs and spleen analyzed for bacteria concentration 18 hours after inoculation.

Statistical evaluation of differences between the experimental groups can be determined by using One-way ANOVA followed by a Tukey' post test.

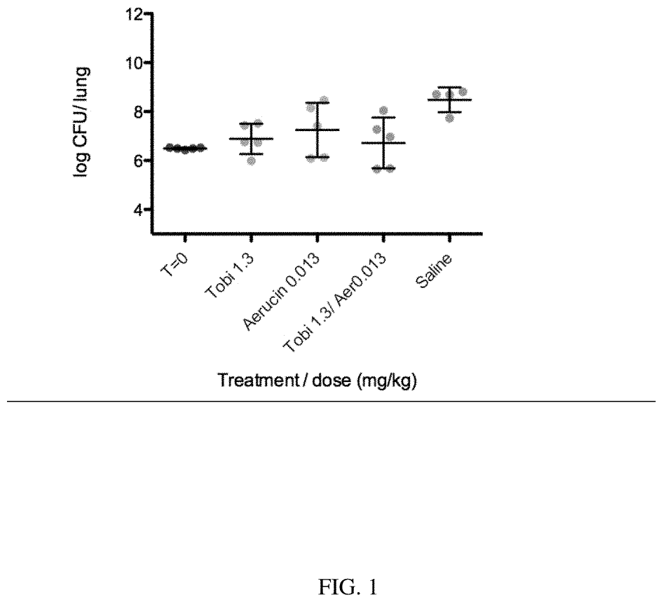

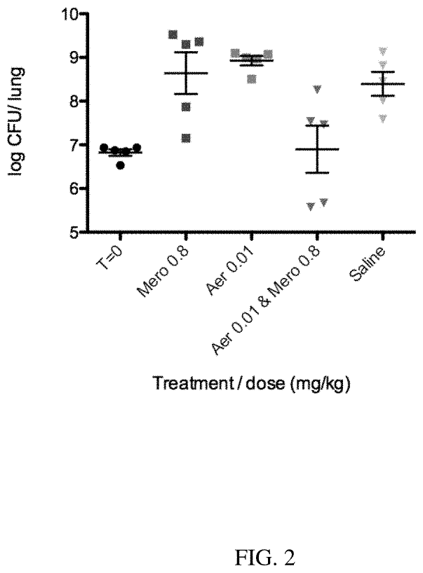

The combination of Aerucin and Tobramycin results in an additive effect, the combination of Aerucin and Meropenem in a synergistic effect (FIG. 1 and FIG. 2).

### Example 2—Anti-Bacterial mAbs in Opsonic Phagocytosis Assay (OPA)

OPA is used to demonstrate killing of Pseudomonas aeruginosa by human neutrophils induced by Aerucin™. The assay is designed to closely simulate the immune response initiated by Aerucin™ in vivo, complement-mediated opsonic phagocytosis of Aerucin™-bound Pseudomonas by neutrophils.

The assay is performed in 96-well microtiter plates with 1% BSA in MEM as the assay diluent. The Leukemia-derived human cell-line HL-60 is used as source of neutrophils. Differentiation of HL-60 cells into neutrophils is induced by addition of 100 mM Dimethylformamide (DMF). Neutrophil morphology is verified by expression of CD11b/Mac-1 marker using FACS. Neutrophils are washed, re-suspended in assay diluent, counted and diluted to a density of 2.5×10⁷ cells/ml. Opsonization is mediated by Rabbit sera complement, diluted in assay diluent for a final dilution factor of 1:60. Freshly grown log-phase Pseudomonas strains are re-suspended and diluted in assay diluent to achieve a final ratio of neutrophils to bacteria of 20:1 to 40:1. Dilutions of Aerucin™ from 5 μg/ml to 100 ng/ml are prepared in assay diluent along with a commercial human IgG1 at 5 μg/ml as negative control. Other negative controls include assay mixtures with certain components (complements, neutrophils, antibodies) absent.

Equal volumes (50 μl) of each component are added to assay wells in the order of: antibodies, complement, neutrophils then bacteria. The components are mixed using a multichannel pipette. Samples at time zero are taken, diluted and plated on agar plates for titer determination. Assay plates are then incubated at 37° C. with shaking for 90 minutes. Samples are again taken, diluted and plated on agar plates.

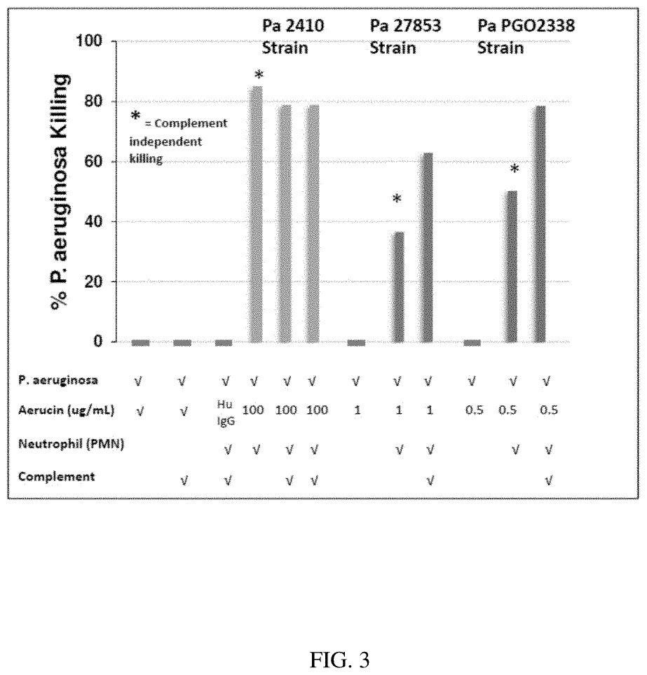

Bacteria titers (CFU/ml) are determined for time-zero and 90-minute samples from colony counts on agar plates after overnight incubation at 37° C. Percent kills are calculated as the difference in titer at 90 minutes from time-zero, divided by the titer at time-zero: 100*(Titer^{T90}−Titer^{T0})/Titer^{T0}. Opsonic phagocytosis by Aerucin™ is indicated by significant increase in killing over negative controls in a dose-dependent manner (FIG. 3).

### Example 3—Testing of Various Anti-Bacterial mAb/Antibiotic Combinations in Opsonic Phagocytosis Assay (OPA)

Aerucin was tested in an in vitro assay for synergistic effect with five antibiotics (Ciprofloxacin, Colistin, Piperacillin, Cefepime, Aztreonam). The assay is performed as described above except that equal volumes (50 μl) of each component are added to assay wells in the order of: antibodies, antibiotic, complement, neutrophils, and then bacteria. Bacteria titers (CFU/ml) are measured directly after mixing all components and after 90 minutes incubation. The read-out of the assay (% kill) is how well the drug or combination of drugs can kill bacteria over these 90 minutes.

Tested concentrations for Aerucin and antibiotics were selected so that each component alone shows little to no killing activity in the OPA assay. Averages from three independent experiments are presented.

In the no-drug control (neither Aerucin nor antibiotic), around 20% of bacteria die over the 90 minutes incubation time. This is within the expected range of values for the no-drug control, where results typically show a minor degree of growth or kill over the 90 minutes. With 50 ng/ml Aerucin, around 5% of bacteria die. Also this value is within the expected range for no observed effect. Each further block of 2 bars represents the % Kill observed with an antibiotic alone, or with an antibiotic in combination with 50 ng/ml Aerucin.

Ciprofloxacin: 0.5 μg/ml Ciprofloxacin does not show bacterial killing. The ‘negative % Kill’ with Ciprofloxacin, representing slight growth over the 90 minutes incubation time, is within the normal variation of this assay for the no-drug control. While Aerucin and Ciprofloxacin by itself do not show bacterial killing at the tested concentration, a combination of both leads to ˜60% killing of bacteria. Aerucin and Ciprofloxacin have a clear synergistic effect.

Colistin at 2 μg/ml shows average killing in the range of 20%. Addition of Aerucin increases the killing to an average of nearly 60%. Aerucin and Colistin may have a synergistic effect.

Piperacillin at 8 μg/ml shows ˜40% killing. Addition of Aerucin improves killing slightly, but consistently.

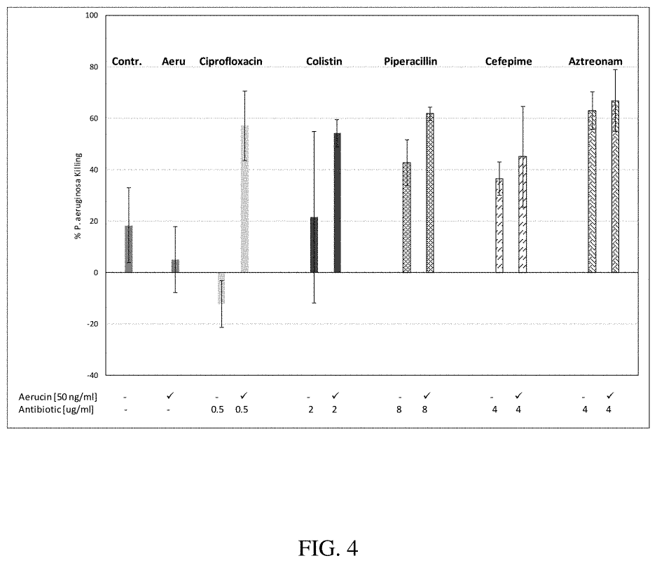

Cefepime and Aztreonam at 4 μg/ml show killing of 40% and 60%, respectively. Addition of Aerucin does not improve the killing efficiency. (FIG. 4)

Minimum inhibitory concentrations (MICs) of the antibiotics used in the OPA assays had been determined upfront (data not shown). Data reported in Example 3 was generated using 2× the MIC for Ciprofloxacin and Colistin, and the antibiotic alone at 2×MIC does not have a clear bactericidal effect (no kill or growth, similar to the ‘no-drug-control’). Data for bactericidal antibiotics Cefepime and Piperacillin was generated with 1×MIC, for Aztreonam (bactericidal) with ½ MIC.

We believe these data demonstrate, across a broad range of antibiotic types, at bacteriostatic or MIC levels, a clear synergistic or complementary effect in the combination with the antibody.

It is understood that the examples and embodiments described herein are for illustrative purposes only and that various modifications or changes in light thereof will be suggested to persons skilled in the art and are to be included within the spirit and purview of this application and scope of the appended claims.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A composition comprising:
an isolated polypeptide that selectively binds to P. aeruginosa mucoid exopolysaccharide; and,
an antibiotic selected from the group consisting of: a carbapenem, a polymyxin a carboxypenicillin, a fluoroquinolone, and an aminoglycoside;
wherein the combination of the polypeptide and antibiotic provides a bactericidal effect greater than either alone.

2. The composition of claim 1, wherein the combination of the polypeptide and antibiotic provides an additive bactericidal effect.

3. The composition of claim 1, wherein the combination of the polypeptide and antibiotic provides a synergistic effect greater an additive bactericidal effect.

4. The composition of claim 1, wherein the combination of the polypeptide and antibiotic provides the same or less than an additive effect, but the overall effect is enhanced by one or more interactions between the antibody and antibiotic, which enhancement is driven by a mode of action not present with either the antibody or antibiotic alone.

5. The composition of claim 1, wherein the polypeptide comprises an amino acid sequence selected from the group consisting of SEQ ID NO: 5 and SEQ ID NO: 7 or a variant having at least 95% identity to SEQ ID NO: 5 or SEQ ID NO: 7.

6-32. (canceled)


SEQUENCE LISTING (42 sequences)

SEQ ID NO: 1 - DNA, 369 bp, Homo sapiens
cagctgcagctgcaggagtcgggccccggactggtgaagcctacggagaccctgtccctcacctgcactgtctctggtggccccatcacctatattaattactactggggctgggtccgccagcccccagggaaggggctggagtggattgggagtatccattatgatgggagcaccttctacaacccgtccctcaagactcgcgtcaccatatcaggagacacgtccaagagcgagttctctgtgaagctgagttctgtgaccgccgcggacacggccgtctattactgtgcgagaacgtattacgatgcttcggggagcccttactttgaccactggggccagggaaccctggtcaccgtctcctca

SEQ ID NO: 2 - DNA, 330 bp, Homo sapiens
cagtctgtgttgacgcagccgccctcagtgtctgcggccccaggacagagggtcaccatctcctgctctggaagcagctccaaccttgggaacaattttgtatcctggtaccagcaactcccaggagcagccccccggctcctcatttatgacaatgataagcgaccctcagggattcctgaccgattctctggctccaagtctggcacgtcagccaccctgggcatcaccgggctccagactggggacgaggccgattattactgcggaacatgggatagcagcctgactgcttatgtcttcggaagtgggaccaaggtcaccgtccta

SEQ ID NO: 3 - DNA, 330 bp, Homo sapiens
cagtctgtgttgacgcagccgccctcagtgtctgcggccccaggacagaaggtctccatctcctgctctggaagcagctccaacattgggaataattatgtatcctggtaccagcagctcccaggaacagcccccaatctcctcatttatgacaataataagcgaccctcagggattccggaccgattctctggctccaagtctggcacgtcagccaccctggacatcaccggactccagagtggggacgaggccgattattactgcggaacatgggatagcagcctgagtacttgggtgttcggcggagggaccaaactgaccgtccta

SEQ ID NO: 4 - DNA, 378 bp, Homo sapiens
cagctgcacctgcaggagtcgggcccaggactagtgaagccttcggagaccctgtccctcacgtgcactgtctctggtggccccatcaccagtaataattactactggggctggatccgccagcccccagggaaggggctggagtggattgggactatctcttataatgggtacacctactacatcccgtccctcaggggtcgagtcaccatatccggagacacgtccaagaaccagttctccctgagggtgaactctgtgaccgccgcagacacggctatgtattactgtgcgagacatgactatagcatgtcgtccggacttactgacaactggttcgacccctggggccagggaaccctggtcaccgtctcctca

SEQ ID NO: 5 - protein, 123 aa, Homo sapiens
QLQLQESGPGLVKPTETLSLTCTVSGGPITYINYYWGWVRQPPGKGLEWIGSIHYDGSTFYNPSLKSRVTISGDTSKSEFSVKLSSVTAADTAVYYCARTYYDASGSPYFDHWGQGTLVTVSS

SEQ ID NO: 6 - protein, 109 aa, Homo sapiens
QSVLTQPPSVSAAPGQRVTISCSGSSSNLGNNFVSWYQQLPGAAPRLLIYDNDKRPSGIPDRFSGSKSGTSATLGITGLQTGDEADYYCGTWDSSLTAYVFGSGTKVTV

SEQ ID NO: 7 - protein, 110 aa, Homo sapiens
QSVLTQPPSVSAAPGQKVSISCSGSSSNIGNNYVSWYQQLPGTAPNLLIYDNNKRPSGIPDRFSGSKSGTSATLDITGLQSGDEADYYCGTWDSSLSTWVFGGGTKLTVL

SEQ ID NO: 8 - protein, 126 aa, Homo sapiens
QLHLQESGPGLVKPSQTLSLTCTVSGGPITSNNYYWGWIRQPPGKGLEWIGTISYNGYTYYIPSLRGRVTISGDTSKNQFSLRVNSVTAADTAMYYCARHDYSMSSGLTDNWFDPWGQGTLVTVSS

SEQ ID NO: 9 - DNA, 21 bp, Homo sapiens
tatattaattactactggggc

SEQ ID NO: 10 - DNA, 49 bp, Homo sapiens
agtatccattatgatgggagcaccttcttacaacccgtccctcaagagt

SEQ ID NO: 11 - DNA, 39 bp, Homo sapiens
acgtattacgatgcttcggggagcccttactttgaccac

SEQ ID NO: 12 - DNA, 39 bp, Homo sapiens
tctggaagcagctccaaccttgggaacaattttgtatcc

SEQ ID NO: 13 - DNA, 21 bp, Homo sapiens
gacaatgataagcgaccctca

SEQ ID NO: 14 - DNA, 33 bp, Homo sapiens
ggaacatgggatagcagcctgactgcttatgtc

SEQ ID NO: 15 - DNA, 39 bp, Homo sapiens
tctggaagcagctccaacattgggaataattatgtatcc

SEQ ID NO: 16 - DNA, 21 bp, Homo sapiens
gacaataataagcgaccctca

SEQ ID NO: 17 - DNA, 33 bp, Homo sapiens
ggaacatgggatagcagcctgagtacttgggtg

SEQ ID NO: 18 - DNA, 21 bp, Homo sapiens
agtaataattactactggggc

SEQ ID NO: 19 - DNA, 48 bp, Homo sapiens
actatctcttataatgggtacacctactacatcccgtccctcaggggt

SEQ ID NO: 20 - DNA, 48 bp, Homo sapiens
catgactatagcatgtcgtccggacttactgacaactggttcgacccc

SEQ ID NO: 21 - protein, 7 aa, Homo sapiens
YINYYWG

SEQ ID NO: 22 - protein, 16 aa, Homo sapiens
SIHYDGSTFYNPSLKS

SEQ ID NO: 23 - protein, 13 aa, Homo sapiens
TYYDASGSPYFDH

SEQ ID NO: 24 - protein, 13 aa, Homo sapiens
SGSSSNLGNNFVS

SEQ ID NO: 25 - protein, 7 aa, Homo sapiens
DNDKRPS

SEQ ID NO: 26 - protein, 11 aa, Homo sapiens
GTWDSSLTAYV

SEQ ID NO: 27 - protein, 13 aa, Homo sapiens
SGSSSNIGNNYVS

SEQ ID NO: 28 - protein, 7 aa, Homo sapiens
DNNKRPS

SEQ ID NO: 29 - protein, 11 aa, Homo sapiens
GTWDSSLSTWV

SEQ ID NO: 30 - protein, 7 aa, Homo sapiens
SNNYYWG

SEQ ID NO: 31 - protein, 16 aa, Homo sapiens
TISYNGYTYYIPSLRG

SEQ ID NO: 32 - protein, 16 aa, Homo sapiens
HDYSMSSGLTDNWFDP

SEQ ID NO: 33 - protein, 112 aa, Homo sapiens
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPLTFGGGTKVEIK

SEQ ID NO: 34 - protein, 116 aa, Homo sapiens
EEQVVESGGGFVQPGGSLRLSCAASGFTFSPYWMHWVRQAPGKGLVWVSRINSDGSTYYADSVKGRFTISRDNARNTLYLQMNSLRAEDTAVYYCARDRYYGPEMWGQGTMVTVSS

SEQ ID NO: 35 - DNA, 336 bp, Homo sapiens
gatgttgtgatgactcagtctccgctctccctgcccgtcacccttggacagccggcctccatctcctgcaggtctagtcaaagcctcgtatatagtgatggaaacacctacttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggcctctcactttcggcggagggaccaaggtggagatcaaa

SEQ ID NO: 36 - DNA, 348 bp, Homo sapiens
gaggagcaggtggtggagtccgggggaggctttgttcagcctggggggtccctgagactctcctgtgcagcctctggattcacctttagtccatactggatgcactgggtccgccaagctccagggaaggggctggtgtgggtctcacgtattaatagtgatgggagcacatactacgcggactccgtgaagggccgattcaccatctccagagacaacgccaggaacacactgtatctgcaaatgaacagtctgagagccgaggacacggctgtgtattactgtgcaagagatcgatactatggccccgaaatgtggggccaagggacaatggtcaccgtctcttca

SEQ ID NO: 37 - protein, 16 aa, Homo sapiens
RSSQSLVYSDGNTYLN

SEQ ID NO: 38 - protein, 7 aa, Homo sapiens
KVSNRDS

SEQ ID NO: 39 - protein, 9 aa, Homo sapiens
MQGTHWPLT

SEQ ID NO: 40 - protein, 5 aa, Homo sapiens
PYWMH

SEQ ID NO: 41 - protein, 16 aa, Homo sapiens
RINSDGSTYYADSVKG

SEQ ID NO: 42 - protein, 8 aa, Homo sapiens
DRYYGPEM